(19) United States
(12) Patent Application Publication
(10) Pub. No.: US 2007/0248716 A1
(43) Pub. Date: Oct. 25, 2007

(54) Method For the Production of a Biological Material Composition Having an Animal Origin

(75) Inventors: Charli Kruse, Herrnburg (DE); Gunter Fuhr, Berlin (DE); Jurgen Rohwedel, Lubeck (DE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Fraunhofer-Gesellschaft Zur Forderung Der Angewandten Forschung E.V., München (DE)

(21) Appl. No.: 10/599,713

(22) PCT Filed: Mar. 4, 2005
(86) PCT No.: PCT/EP05/02313

§ 371 Date: Oct. 25, 2006

(30) Foreign Application Priority Data
Apr. 8, 2004 (DE) 10 2004 017 484.9

(51) Int. Cl. A23K 1/00; A23L 1/00; C12N 5/02; C12N 5/06

(52) U.S. Cl. 426/2; 426/641; 435/377; 435/378

(57) ABSTRACT

A method for producing a biological material composition of animal, non-human origin, especially as feed or food, is described. The method includes an aggregation of stem cells from differentiated exocrine glandular tissue of an animal organism to organoid bodies, and a preparation of the material composition from the organoid bodies.

The invention relates to methods for producing biological material compositions of animal origin, especially as feed or food, biological material compositions containing especially proteins, fats and/or carbohydrates of animal origin and their use.

The production of biomass as feed for the nourishment of animals is one of the most important challenges of modern animal husbandry. There are important problems in the conventional production of feeds of animal origin, especially for domestic animals or working animals, firstly in the availability of biological starting materials, whose provision and obtention constitutes a great economic expense and causes ethical problems, and secondly given the danger that pathogens are introduced into the food chain and may be transmitted to humans.

There are also similar problems in the production of food of animal origin for nourishing humans. The so-called meat production (including raising, animal husbandry and slaughter) takes place today under conditions that are ethically highly problematic, especially for the well-being of mammals.

There is great interest in an increasing production of feed and food on the basis of animal starting materials in order to, e.g., do justice to the requirements of an increasing population and for food in hunger areas. Neither an expanded animal production nor a greater catching of fish furnishes permanent solutions for this.

The invention has the object of providing improved methods for the production of a biological material composition of animal origin, especially as feed or food, with which the problems of the traditional feed or food production are overcome and that in particular make it possible to produce feed or food that are equivalent or identical to the previously obtained animal products in their composition and usability. Another aspect of the object of the invention is to provide a corresponding biological material composition for feed or nourishing purposes in the production of which the disadvantages of the traditional animal production are avoided, that is, no organisms have to be raised, fattened and processed to meat products.

These objects are achieved by methods having the features of Claim 1 and a material composition having the features of Claim 21. Advantageous embodiments and applications of the invention are derived from the dependent claims.

As concerns the methods, the above-mentioned object is achieved by an aggregation of stem cells that were isolated from differentiated exocrine glandular tissue of an animal (non-human) organism to three-dimensional cell aggregates and by a subsequent preparation of the desired material composition on the basis of the cell aggregates. The cell aggregates are also designated in the following as organoid bodies. A special advantage of this method consists in the complete decoupling of the biomass production from the traditional animal production, in which, however, a material, structural and particular taste individuality can be imparted to the produced material composition as a function of the animal organism from which the stem cells were isolated.

The inventors observed that the adult stem cells isolated from the exocrine glandular tissue are pluripotent and exhibit a high capacity for division and a strong growth. The production of biomass does not take place naturally by the growth of the animal but rather synthetically, in particular in the preparation of the material composition in so-called in-vitro laboratory cultures.

The exocrine glandular tissue used in accordance with the invention can stem from a non-human adult organism, juvenile organism or fetal organism, preferably a postnatal organism. The concept “adult” as used in the present application thus refers to the development stage of the starting tissue and not to that of the donor organism from which the tissue stems. “Adult” stem cells are non-embryonic stem cells.

The exocrine glandular tissue is preferably isolated from a salivary gland, lachrymal gland, sebaceous gland, sweat gland, from glands of the genital tract including the prostate, or from gastrointestinal tissue, including the pancreas, or from secretory tissue of the liver. In a highly preferred embodiment, acinar tissue is concerned. A most preferred embodiment concerns acinar tissue from the pancreas, the parotid gland or the mandibular salivary gland.

A substantial advantage of the method of the invention is that the stem cells can be obtained from living donor animals, e.g. from salivary glands of swine without the donor animal having to be slaughtered. This is especially advantageous from ethical standpoints as well as in view of the possibility of further observing the donor animal for possible diseases.

In general, according to the invention the material composition can be formed directly from the organoid bodies. However, a preferred embodiment of the invention provides that the organoid bodies grow to larger bodies designated in the following as tissue bodies after the aggregation, e.g., in a suspension culture or an adhesion culture with the supplying of nutrients. The inventors observed that the stem cells isolated from the exocrine glands form organoid bodies that exhibit, when supplied with nutrients, a strong growth to tissue bodies with diameters of a few millimeters or more. The preparation of the material composition on the basis of the tissue bodies has the advantage of an increased effectiveness in the production of biomaterials. The organoid bodies contain no organs or a functioning nervous system but rather consist of tissue layers of different cellular compositions.

If, according to a first variant of the invention, the aggregation of the stem cells and/or the growth of the organoid bodies take place in a culture medium without an additive that might be able to influence the differentiation of the cells of the organoid body, organoid bodies or tissue bodies having the composition of different cell types advantageously grow, which cell types jointly contribute to the structure and the usability, in particular to the taste of the desired material composition. On the other hand, if the aggregation and/or the growth take place in the presence of at least one additive that influences the differentiation, organoid bodies or tissue bodies with one or more preferred cell types can be formed. Different cell types stemming from different donor animals can be combined in order to achieve certain properties of food or taste.

According to a preferred embodiment of the invention the organoid bodies or the tissue bodies that grew from them are combined to a composite (formed body). If the cells live in the composite, a further growth of the composite up to the desired size as feed or food can advantageously take place. On the other hand, if the cells in the composite have died off and no longer grow, advantages for the further processing of the material composition can result.

According to preferred variants of the invention the formation of the composite therefore comprises one or more of the following steps. Firstly, a growing together of the participating organoid bodies or tissue bodies to the composite can be provided, which can then advantageously continue to grow. Secondly, an adherent adhesion of the participating organoid bodies or tissue bodies can be provided, where in this instance also a further growth can be provided. A compression of the formed body and therewith a certain structure can be achieved by pressing the organoid bodies or tissue bodies together. Finally, a transfer of the organoid bodies or tissue bodies to a biocompatible carrier substrate of a digestible material can be provided on which a further preparation or forming of the material composition takes place.

According to a further preferred embodiment of the invention the composite can be subjected to a shaping by an imprinting device. In this instance, advantages for the presentation of the feed or food for their consumption may result. The imprinting device can comprise a stamping device with an imprinting surface, e.g., by means of a specially formed cultivation substrate such as e.g., the carrier substrate, or can comprise a flexible bag forming a receiving casing.

According to a further modification of the method according to the invention a structural adjustment can be provided in the preparation of the material composition. As a result thereof, the material composition can be formed with a desired consistency such as, e.g., solid, tough, loose or the like, which can be advantageous in particular for the use as food. The structural adjustment can take place, e.g., by said mechanical compression. Alternatively, a structural adjustment is realized by the effect of electrical fields, in particular if the tissue bodies contain muscle cells that can be stimulated electrically. The composite is stimulated by an electrical field so that the muscle cells form a structure similar to that of muscle flesh.

The organoid bodies are subjected with particular preference to a differentiation that results in at least one of the following cell types comprising muscle cells, connective tissue cells, fat cells and enzyme-producing cells. In this instance, the organoid bodies or corresponding tissue bodies preferably contain the differentiated cell type. The differentiation can take place in general by the addition of known differentiation factors. However, an addition of already differentiated, in particular autologous cells that initiate or influence the further differentiation in the organoid body or tissue body takes place especially preferably in the cultivation of the organoid bodies or in the growth of the tissue bodies.

If the biological material composition is prepared from different groups of organoid bodies or tissue bodies that were differently differentiated, advantages can result for the further use of the material composition, especially as regards the nutrient composition, the structure and the taste. As an alternative or a supplement, flavoring substances like those known from food technology can be added to the material composition in accordance with the invention.

Independent subject matter of the present invention represents a biological material composition such as, e.g., a formed body or composite of biological material, especially with proteins, carbohydrates and/or fats, that was produced by the method of the invention and is used as feed or food such as, e.g., synthetic meat product.

Further details and advantages of the invention are explained in the following with reference made to the attached drawings.

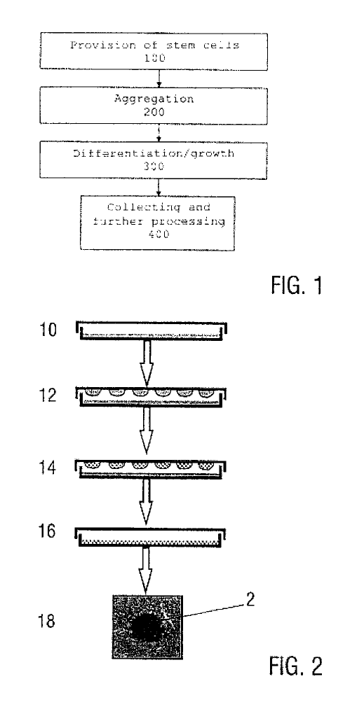

FIG. 1 shows a flowchart for illustrating an embodiment of the method in accordance with the invention.

FIG. 2 shows an illustration of the formation of primary organoid bodies.

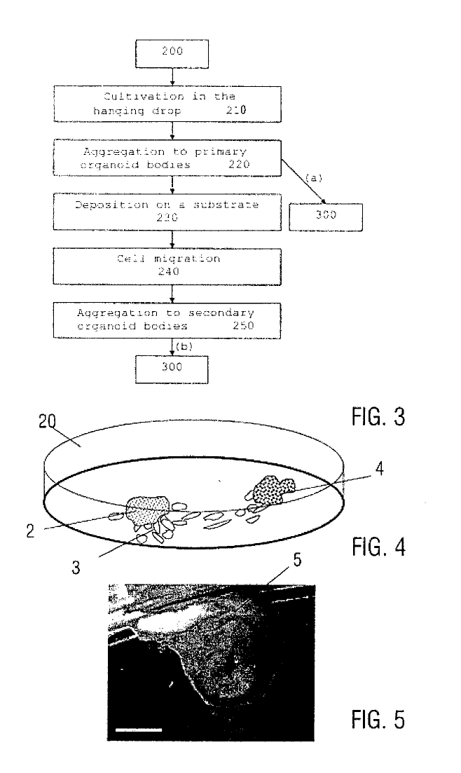

FIG. 3 shows a flowchart for illustrating further details of the method in accordance with the invention.

FIG. 4 shows the formation of secondary organoid bodies.

FIG. 5 shows a photograph of a tissue body.

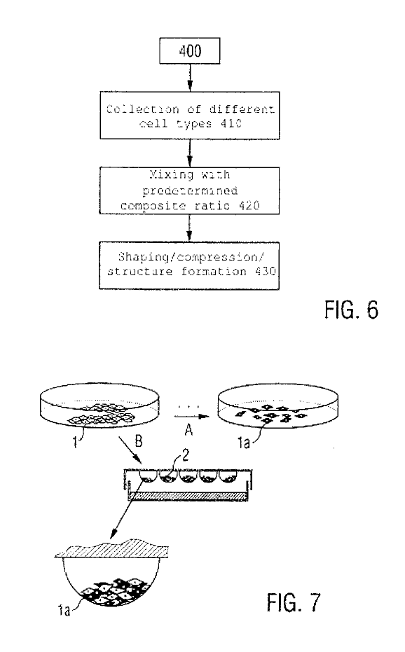

FIG. 6 shows a flowchart with further details of the method in accordance with the invention.

FIG. 7 shows a schematic representation for illustrating the cultivation of organoid bodies.

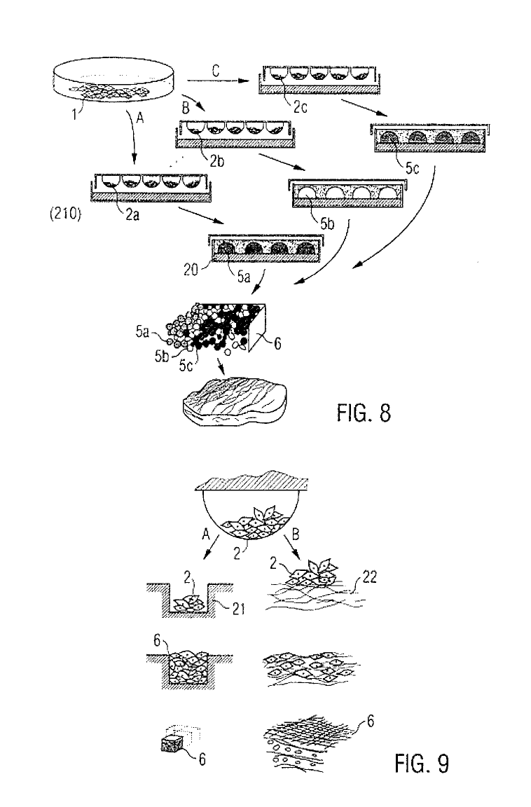

FIG. 8 shows a schematic representation with further details of an embodiment of the method in accordance with the invention.

FIG. 9 shows illustrations of the preparation steps in accordance with different embodiments of the invention.

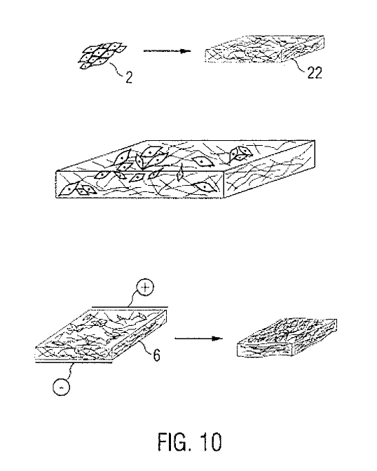

FIG. 10 shows an illustration of an electric structural adjustment in a material composition in accordance with the invention.

In order to produce a composition of biological cells and/or tissue in accordance with the invention, at first the stem cells isolated from a donor organism are provided (step 100) according to FIG. 1, then their aggregation to organoid bodies (step 200) that are subjected to a differentiation and/or to a growth (step 300) and finally to a collection and further processing (step 400). Although steps 100 and 200 substantially comprise the aggregation step in accordance with the invention, whereas steps 300 and 400 substantially represent the preparation step in accordance with the invention and these two complexes are discussed separately in the following, it is stressed that partial steps of the preparation take place already within the framework of the aggregation and, vice versa, partial steps of the aggregation may be realized at first within the framework of the preparation. Thus, e.g., the differentiation of the organoid bodies can begin within the framework of the aggregation already or isolated stem cells may aggregate with organoid bodies that had already been pre-differentiated.

The embodiments of the invention explained in the following refer by way of example to the production of the biological material composition from stem cells removed from rats or swines. However, the implementation of the invention is not limited to these animal types but rather the corresponding methods can be realized with all non-human organisms with differentiated exocrine glands with acinar tissue, thus, e.g., with fish that have acinar tissue on the pancreas gland or mammals such as, e.g., cattle or sheep.

(I) Aggregation of Stem Cells from Differentiated Exocrine Glandular Tissue of an Animal Organism to Organoid Bodies and/or Tissue Bodies

According to the scheme shown in FIG. 2, in order to obtain the cells acinar tissue, preferably from a salivary gland or the salivary gland of the abdomen (pancreas), is taken in culture in a mechanically and enzymatically comminuted form (step 10 in FIG. 2). Contrary to the indications of Bachem et al., Gastroenterol. 115:421-432 (1998) and Grosfils et al., Res. Comm. Chem. Path. Pharmacol. 79:99-115 (1993), no tissue blocks are cultivated out of which cells are supposed to grow but rather the tissue is comminuted more strongly, with the proviso that the cell aggregates of the acini remain intact to a very large extent.

These cells and cell aggregates are cultivated for several weeks in culture vessels. Every 2 to 3 days the medium is changed, all differentiated cells being removed at this time. The cells persisting in culture are non-differentiated cells with an unlimited capacity for division.

Similar cells have been isolated and described under the same conditions from the pancreas and designated as a type of myofibroblasts or pancreatic star cells (Bachem et al., 1998). However, in contrast to the cells of the present invention an unlimited ability to divide could not be observed. Furthermore, these cells were also not able to be passaged in an unlimited manner without loosing vitality.

In a second step (12) approximately 400 to 800 cells are cultivated in 20 μl medium in hanging drops. To this end, the drops are placed on covers of bacteriological Petri dishes, turned upside down and placed over the Petri dish filled with medium so that the drops hang downward.

As a result of this type of cultivation cell aggregates (14) designated as organoid bodies form within 48 h and are transferred into a suspension culture for approximately 6 days (16). The partial view (18) in FIG. 2 shows a microscopic photograph of such an organoid body 2.

The various possibilities of providing organoid bodies as starting materials for further preparation are compiled with further details in FIG. 3. The aggregation to the so-called primary organoid bodies (step 220) takes place after the above-described cultivation of the isolated stem cells in hanging drops (step 210), which primary organoid bodies can be subjected directly to the differentiation and the growth (step 300 in FIG. 1).

As an alternative, the depositing of the primary organoid bodies on a substrate takes place at first for creating an adhesion culture (see also FIG. 4). The inventors observed that the primary organoid bodies growing in suspension culture can form new organoid bodies in adhesion culture. The formation of a monolayer (step 240) follows on the substrate by a cell migration and from this monolayer the aggregation to the so-called secondary organoid bodies (step 250). The latter can be subjected directly to the further differentiation and/or the growth (step 300).

The formation of the secondary organoid bodies is also illustrated in FIG. 4. Primary organoid bodies 2 first form a monolayer on the substrate of the adhesion culture, such as, e.g., on the bottom of culture dish 20, by a migration or growth of cells 3 from which monolayer secondary organoid bodies 4 then grow out. A further multiplication of the biomaterial is created with the cultivation of primary organoid bodies 2 to secondary organoid bodies.

Tissue bodies can grow from each of the primary or secondary organoid bodies 2, 4 during further cultivation. FIG. 5 shows by way of example tissue body 5 grown in the adhesion culture (the white line corresponds to a length of 2 mm in the original).

### Exemplary Embodiments for the Isolation and Aggregation of Stem Cells

The isolation and aggregation of stem cells is explained in detail in the following non-limiting examples.

The general working instructions customary for methods for cultivating animal cells and in particular mammalian cells are to be observed. A sterile environment in which the method is to be carried out is to be observed in any case, even if no further description for this is given. The following buffers and media were used:

| | |
|---|---|
| HEPES stock | 2.383 g HEPES per 100 ml A. bidest. |
| solution (pH 7.6) | |
| HEPES Eagle's | 90 ml modified Eagle's Medium (MEM) |
| Medium (pH 7.4) | 10 ml HEPES stock solution |
| Isolation medium | 32 ml HEPES Eagle's Medium |
| (pH 7.4) | 8 ml 5% BSA in A. bidest. |
| | 300 μl 0.1 M CaCl₂ |
| | 100 μl trasylol (200,000 KIU) |
| Digestion medium | 20 ml Isolation medium |
| (pH 7.4) | 4 ml collagenase (collagenase NB 8 from Serva) |
| Inkubation medium | Dulbecco's modified Eagle's Medium (DMEM) |
| Nutrient medium | Dulbecco's modified Eagle's Medium (DMEM) |
| | DMEM + 4500 mg/l glucose + L-glutamine − |
| | pyruvate + 20% FCS (inactivated) + 1 ml/100 ml |
| | pen/strep (10000 U/10000 μg/ml) or |
| | DMEM + 10% autoplasma + 1 ml/100 ml |
| | pen/strep, warm to 37° C. before use |
| Differentiation | 380 ml DMEM |
| medium | 95 ml 30 min at 54° C. inactivated FCS |
| | 5 ml glutamine (GIBCO BRL) |
| | 5 ml (3.5 μl β-mercaptoethanol per 5 ml PBS) |
| | 5 ml nonessential amino acids (GIBCO BRL) |
| | 5 ml penicillin/streptomycin (GIBCO BRL) |
| | (10000 U/10000 μg/ml) |

Instead of fetal calf serum (FCS) in the nutrient medium and differentiation medium autoplasma, or, less preferably, autoserum of the tissue donor can also be used as an option.

Instead of the DMEM medium used, the nutrient medium may also contain another known base medium suitable for the cultivation of eukaryotic cells, especially mammalian cells, as base medium in which the differentiated cells die and the desired stem cells propagate. The isolation medium, incubation medium and differentiation medium may also contain another customary and suitable base medium.

The following examples 1 and 2 describe in detail two working protocols for isolating and cultivating adult pluripotent stem cells from acinar tissue of the pancreas. Example 3 describes a corresponding protocol for the isolating from acinar tissue of the salivary gland.

### EXAMPLE 1

1. Preparation of the Tissue and Isolation of the Cells

10 ml digestion medium are injected slowly and without air bubbles into the Ductus pancreaticus of 2-3-year old rats with a syringe and a blunt cannula in the rat. This inflates the entire pancreas and it can thus be better prepared for removal. The pancreas is then transferred into a beaker glass and 5 ml more digestion medium are added to it. After the fatty tissue and lymph nodes have been removed, the tissue is comminuted very finely in the beaker glass with fine shears, fatty tissue floating on top is removed by suction and the suspension is subsequently gassed 1 min with Carbogen (repeat if necessary) and incubated in an agitator at 200 cycles/min for 20 min at 37° C. covered with aluminum foil. Then, the medium is carefully removed by suction, the tissue comminuted again with shears and the tissue pieces washed twice with 10 ml isolation medium each time and 5 ml digestion medium is again added to the tissue.

After another gassing with Carbogen for approximately 1 minute and incubation for 15 min at 37° C. in an agitator at 200 cycles/min, the tissue pieces are comminuted by successively being drawn up in a 10 ml, 5 ml, 2 ml and 1 ml glass pipette and pressed through a monolayer filter tissue. The cells individualized in this manner are now washed five times in incubation medium (37° C.), gassed with Carbogen and centrifuged each time for 5 min at 90 g. The last pellet obtained is re-suspended in incubation medium, gassed and distributed onto tissue culture dishes.

2. Cultivation of the Cells

The tissue culture dishes with the isolated cells are cultivated in an incubator at 37° C. and 5% CO₂. The medium is replaced every 2-3 days at which time all differentiated cells are removed.

On the seventh day in culture the cells are passaged with a solution consisting of 2 ml PBS, 1 ml trypsin and 2 ml incubation medium, during which the cells separate from the bottom of the culture dish. The cell suspension is centrifuged for 5 minutes, the supernatant removed by suction and the cells are re-suspended in 2 ml incubation medium, transferred to a medium cell culture bottle and 10 ml incubation medium are added thereto. The medium is replaced every three days.

On the fourteenth day in culture, the cells are passaged again but this time with 6 ml PBS, 3 ml trypsin and 6 ml incubation medium. The cell suspension is centrifuged for 5 minutes, the supernatant removed by suction and the cells re-suspended in 6 ml incubation medium, transferred to three medium cell culture bottles and 10 ml incubation medium added to each one.

The cells are cultivated further and passaged and seeded until the cells achieve a semi-confluent to confluent state.

### EXAMPLE 2

Pancreas acini were obtained from male Sprague-Dawley rats (20-300 g) that had been narcotized (CO₂) and exsanguinated via the dorsal aorta. A cannula was introduced transduodenally into the pancreatic duct and 10 ml digestion medium containing HEPES Eagle's Medium (pH 7.4), 0.1 mM HEPES buffer (pH 7.6), 70% (vol./vol.) modified Eagle's Medium, 0.5% (vol./vol.) trasylol (Bayer AG, Leverkusen, Germany), 1% (wt./vol.) Bovine serum albumin), 2.4 mM CaCl₂ and collagenase (0.63 P/mg, Serva, Heidelberg, Germany) were injected into the pancreas from the rear.

Prior to the removal, the pancreas was partially freed of adhering fatty tissue, lymph nodes and blood vessels. Then, healthy pancreatic tissue was taken in digestion medium (at 20° C., lesser metabolism), the pancreatic tissue very finely comminuted with shears, fatty tissue floating on top removed by suction and the tissue suspension gassed with Carbogen (Messer, Krefeld, Deutschland) without the jet passing into the medium with the cells (reduction of mechanical stress) and adjusted therewith to pH 7.4. The suspension was then incubated in a 25 ml Erlenmeyer flask (covered with aluminum foil) under constant agitation (150-200 cycles per minute) at 37° C. in 10 ml digestion medium. After 15-20 minutes the fat floating on top and the medium were removed by suction and the tissue was again comminuted and rinsed with medium without collagenase (repeat procedure at least twice, preferably until cell fraction is transparent), whereupon digestion medium was added and another gassing was performed for approximately 1 minute with Carbogen. A digestion with collagenase followed again for 15 minutes at 37° C. in an agitator using the same buffer. After the digestion the acini were dissociated by successively drawing them up and ejecting through 10 ml, 5 ml and 2 ml glass pipettes with narrow openings and filtered through a single-layer nylon mesh (Polymon PES-200/45, Angst & Pfister AG, Zurich, Switzerland) with a mesh size of approximately 250 μm. The acini were centrifuged (at 37° C. and 600-800 rpm in a Beckman GPR centrifuge, corresponds to approximately 90 g) and further purified by being washed in incubation medium containing 24.5 mM HEPES (pH 7.5), 96 mM NaCl, 6 mM KCl, 1 mM MgCl₂, 2.5 mM NaH₂PO₄, 0. mM CaCl₂, 11.5 mM glucose, 5 mM sodium pyruvate, 5 mM sodium glutamate, 5 mM sodium fumarate, 1% (vol./vol.) modified Eagle's Medium, 1% (wt./vol.) bovine serum albumin, equilibrated with Carbogen and adjusted to pH 7.4. The washing procedure (centrifugation, removal by suction, re-suspension) was repeated five times. Unless otherwise indicated, the work was performed at approximately 20° C. in the above isolation.

The acini were re-suspended in incubation medium and cultivated at 37° C. in a moist atmosphere with 5% CO₂. The acinar tissue died rapidly (within two days) and the dying differentiated cells separated from the adjacent cells without damaging them (gentle isolation) and the stem cells that were not dying sank to the bottom, to which they adhered. The differentiated acini are not capable of doing this. The incubation medium was replaced for the first time on the second or third day after the seeding, during which a large part of the freely floating acini and acinar cells was removed. At this time the first stem cells and/or their precursors had settled on the bottom and began to divide. The medium replacement was repeated thereafter on every third day and differentiated acinar pancreatic cells were removed at each medium replacement.

On the seventh day in culture the cells were passaged with a solution consisting of 2 ml PBS, 1 ml trypsin (+0.05% EDTA) and 2 ml incubation medium, during which the cells separated from the bottom of the culture dish. The cell suspension was centrifuged for 5 minutes at approximately 1000 rpm (Beckmann GPR centrifuge), the supernatant removed by suction and the cells were re-suspended in 2 ml incubation medium and transferred to a medium cell culture bottle to which 10 ml incubation medium were added.

On the fourteenth day in culture the cells were passaged again but this time with 6 ml PBS, 3 ml trypsin/EDTA and 6 ml incubation medium. The cell suspension is centrifuged for 5 minutes at 1000 rpm, the supernatant removed by suction and the cells are re-suspended in 6 ml incubation medium, transferred to 3 medium cell culture bottles and 10 ml incubation medium added to each one.

On day 17 a third passage took place to a total of 6 medium cell culture bottles and on day 24 a fourth passage to a total of 12 medium cell culture bottles. Now at the latest all primary cells except for the stem cells had been removed from the cell culture.

The stem cells can be cultivated further and passaged and seeded as often as desired. The seeding preferably takes place at a density of 2-4×10⁵ cells/cm² in the incubation medium.

### EXAMPLE 3

The isolation and cultivation from exocrine tissue of the parotid gland took place analogously to the pancreas protocol with the following deviations:

1. The exocrine tissue of the parotid gland was a mixture of acinar tissue and tubular tissue.

2. Since salivary glands contain less proteases and amylases than the pancreas, it is possible to store salivary gland tissue for a while in a refrigerator at approximately 4° C. without the tissue being damaged too much. In the concrete exemplary case the storage time was 15 h and entailed no disadvantageous consequences for the isolation of the desired stem cells.

The following examples 4 and 5 describe in detail two working protocols for producing organoid bodies and differentiated cells.

### EXAMPLE 4

The undifferentiated cells are trypsinated with a solution consisting of 10 ml PBS, 4 ml trypsin, 8 ml differentiation medium and centrifuged off for 5 minutes. The resulting pellet is re-suspended in differentiation medium in such a manner that a dilution of 3000 cells per 100 μl medium is adjusted. The cells are subsequently well suspended again with a 3 ml pipette.

The cover is removed from bacteriological Petri dishes, which had previously been coated with 15 ml PBS (37° C.) per plate, and inverted. Approximately fifty 20 μl drops were placed with the aid of an automatic pipette on a cover. The cover is then rapidly inverted and placed on the Petri dish filled with differentiation medium so that the drops hang downward. The Petri dishes are subsequently carefully placed in an incubator and incubated for 48 h.

Then, the cells that had aggregated in the hanging drops and are designated organoid bodies herein are transferred from four covers at a time into one bacteriological Petri dish with 5 ml incubation medium with 20% FCS and cultivated for another 96 h.

The organoid bodies are now carefully collected with a pipette and transferred into cell culture vessels coated with 0.1% gelatin and containing differentiation medium. In an especially preferred embodiment of the method 6 cm Petri dishes coated with 0.1% gelatin into which 4 ml differentiation medium had been placed and that were subsequently each loaded with 6 organoid bodies are used as culture vessels. Another preferred culture vessel are chamber slides coated with 0.1% gelatin into which 3 ml differentiation medium had been placed and that were subsequently each loaded with 3-8 organoid bodies. In addition, 24-well microtiter plates that were coated with 0.1% gelatin and into which 1.5 ml per well differentiation medium had been placed and that were subsequently loaded with 4 organoid bodies each may also be used.

Cultivated in this manner, the differentiation capacity of the cells into the organoid bodies is activated and the cells differentiate into cells of the three germ layers mesoderm, entoderm and ectoderm. The cells can be stored and cultivated as organoid bodies as well as individual cells and retain their pluripotency.

### EXAMPLE 5

Stem cells after the 42nd day of cultivation were preferably used for the induction of the differentiation. The use of stem cells after the 3rd or 4th passage or of cells that had been stored at the temperature of liquid nitrogen for 12-18 months was also possible without problems.

At first, the cells were transferred into differentiation medium with the composition indicated above and adjusted to a density of approximately 3×10⁴ cells/ml, e.g., by trypsin treatment of a stem cell culture in nutrient medium, 5-minute centrifugation at 1000 rpm and re-suspension of the pellet in differentiation medium and dilution to the extent required.

Subsequently, approximately 50 20-μl drops (600 cells/20 μl) were placed on the inside of the cover of a bacteriological Petri dish using a 20-ml pipette (plugged tips) and the cover was carefully inverted onto the Petri dishes filled with PBS so that the drops hung downward. A new tip was used for each cover. The Petri dishes were subsequently carefully placed into the incubator and incubated for 48 h at 37° C.

Then, the aggregated cells in the hanging drops, the organoid bodies (OB), were transferred from four covers at a time into one bacteriological Petri dish with 5 ml incubation medium with 20% FCS (hold cover obliquely and rinse the organoid bodies off with approximately 2.5 ml nutrient medium) and cultivated for another 5-9 days, preferably 96 h.

The organoid bodies were now carefully collected with a pipette and transferred into cell culture vessels coated with 0.1% gelatin and containing differentiation medium. The organoid bodies now multiplied and grew in partially individual cell colonies that were again able to be multiplied, isolated and multiplied. In an especially preferred embodiment of the invention 6 cm Petri dishes coated with 0.1% gelatin were used as culture vessels into which 4 ml differentiation medium had been placed and they were each loaded with 6 organoid bodies. Another preferred culture vessel was chamber slides, coated with 0.1% gelatin into which 3 ml differentiation medium had been placed and that were each subsequently loaded with 3-8 organoid bodies, and Thermanox plates (Nalge Nonc International, USA) for electron microscopic studies. Another alternative was 24-well microtiter plates coated with 0.1% gelatin into each of which 1.5 ml differentiation medium per well had been placed and that were subsequently each loaded with 4 organoid bodies.

In a preferred embodiment of the method, organoid bodies were cultivated approximately 7 weeks in the gelatin-coated 6 cm Petri dishes and thereafter individual organoid bodies were cut out with the Microdissector (Eppendorf, Hamburg, Germany) according to the instructions of the manufacturer and then transferred, e.g., onto fresh 6 cm Petri dishes, chamber slides or Thermanox plates.

(II) Preparation of the Material Composition from the Organoid Bodies and/or Tissue Bodies

In general, the steps of the collecting and further processing (step 400) can comprise the partial steps shown in FIG. 6. According to partial step 410 the collecting (“harvesting”) of organoid bodies or tissue bodies takes place with optionally differently differentiated cell types (step 410). The different cell types are subsequently mixed in the desired composite ratio (step 420). Finally, the shaping, compression and/or structural formation on the compound take place (step 430).

The provision of the differently differentiated cell types is shown in FIG. 7 by way of example. In this variant of the method of the invention organoid bodies 2 are subjected to a differentiation under the action of pre-differentiated individual cells. To this end, at first differentiated stem cells 1a are formed from the primarily isolated stem cells 1 (arrow A). The differentiated stem cells 1a comprise, e.g., muscle cells. Organoid bodies 2 are formed in parallel in hanging drops from primary isolated stem cells 1 in accordance with the above-cited principles (arrow B). Muscle cells 1a are added into the hanging drops (see enlarged partial image), where they initiate a differentiation of organoid bodies 2 to muscle cells.

FIG. 8 schematically illustrates a method for producing, in accordance with the invention, a meat-like tissue composition 6. Primarily isolated stem cells 1 comprise a stem cell culture obtained in accordance with the principles described above that can be propagated repeatedly and contains pluripotent or at least multipotent cells. Starting from the primarily isolated stem cells 1, the provision of suspension cultures and the cultivation in hanging drops take place at first (see FIG. 3, step 210). Three suspension cultures are shown by way of example in FIG. 8 that result in the course of the further process in different cell types that are to be combined at the end to desired composite 6. For example, organoid bodies 2a are produced from muscle cells in the first suspension culture (arrow A) whereas in the remaining suspension cultures (arrows B and C), for example, organoid bodies 2b are produced from fat cells and 2c from connective tissue cells. A few up to a few hundred or even a few thousand stem cells 1 are transferred into the hanging drop culture on each path A, B and C. In order to form the drop culture different cultivation media are used containing, e.g., molecular differentiation factors or differentiated cells (see FIG. 7). The drop size is maintained constant by creating a saturated atmosphere in the environment of the drop culture or in a known manner by supplying further culture medium.

After the organoid bodies 2a, 2b, 2c have been formed and have a diameter of, e.g., a few hundred micrometers, the organoid bodies are transferred into the culture dishes 20 in adhesion cultures. Further growth to tissue bodies 5a, 5b and 5c takes place there. Since the cultivation media in the culture dishes 20 contain different signal factors, the tissue bodies 5a, 5b and 5c have correspondingly differentiated tissue. Alternatively, a growth with one cell type mixture can be provided.

When the tissue bodies 5a, 5b and 5c have attained the size of, e.g., a few millimeters they are collected (step 400, see also FIG. 1) and combined, so that a loose compound with a predetermined quantitative composition of the tissue bodies 5a, 5b and 5c is produced. This compound is then transferred into the desired material composition of the composite 6 with the aid of mechanical pressure forces, ultrasound, mechanical vibrations. Cross-linking agents such as, e.g., gels, collagen fibers, flavoring substances, food dyes or the like can be optionally added to the compound.

The composite 6 has typical dimensions of, e.g., a few 100 μm to a few millimeters but may also be larger. A plurality of composites may be combined to the desired size in the production of food.

FIG. 9 schematically illustrates the shaping of the biological composite produced in accordance with the invention using the example of different method variants.

According to a first variant (arrow A), a plurality of organoid bodies 2 or appropriately grown tissue bodies are placed in an imprinting device formed in this example by a cultivation substrate 21 with a given surface structure. A growth and multiplication of the cell material take place on the structured cultivation substrate 21. A certain differentiation can be induced during the cell growth thereby by surface-immobilized molecular growth factors (e.g., proteins). When the surface structure of the cultivation substrate 21 has been filled, the composite 6 can be removed and optionally formed further or combined with other composites (partial image bottom left).

In the alternative variant (arrow B), the organoid bodies are placed on a carrier substrate 22 in the form of a three-dimensional matrix consisting of digestible substances such as, e.g., polymers, biopolymers or the like. During growth the cells of the organoid body 2 migrate into carrier device 22. Accordingly, different base building blocks can be created with a band-, strip- or layer-shaped carrier device that can be combined to a larger composite 6 (partial image bottom right).

FIG. 10 shows, by way of example, the structure adjustment on a layer-shaped carrier substrate 22 that is covered, analogously to variant B in FIG. 9, with organoid bodies or tissue bodies consisting primarily of muscle cells. When the carrier substrate 22, that comprises, e.g., a three-dimensional collagen fleece of autologous collagen, has been completely penetrated and covered, the composite 6 is subjected to a current pulse, whereupon the muscle cells in the composite 6 contract counter to the forces of the collagen matrix. An inner tension is adjusted that is associated with an increased strength of the composite.

The features of the invention disclosed in the above description, the claims and the drawings can be significant individually as well as in combination for realizing the invention in its various embodiments.

1. A method for producing a biological material composition of animal, non-human origin, for use as feed or food, said method comprising:
aggregation of stem cells from differentiated exocrine glandular tissue of an animal organism to form organoid bodies, and
preparation of the material composition from the organoid bodies.

2. The method according to claim 1, wherein the aggregation of the stem cells takes place in a culture medium without an additive that influences a differentiation of cells.

3. The method according to claim 1, wherein the aggregation of the stem cells takes place in a culture medium containing at least one additive that influences a differentiation of the stem cells during the aggregation.

4. The method according to claim 1, wherein the aggregation of stem cells results in primary organoid bodies, the preparation of the material composition comprising a formation of secondary organoid bodies from the primary organoid bodies.

5. The method according to claim 1, wherein the preparation of the material composition comprises a growth of the organoid bodies to form tissue bodies.

6. The method according to claim 4 wherein the formation of the secondary organoid bodies takes place in a culture medium without an additive that influences a differentiation of cells.

7. The method according to claim 4, wherein the formation of the secondary organoid bodies takes place in a culture medium that contains at least one additive by way of which a plurality of the cells belonging to an organoid body or tissue body is differentiated to a certain cell type.

8. The method according to claim 3, wherein the differentiation provides at least one of the following cell types: muscle cells, cartilage cells, connective tissue cells, fat cells and enzyme-producing cells.

9. The method according to claim 3, wherein the differentiation is influenced by an addition of differentiated cells.

10. The method according to claim 9, wherein the differentiation is influenced by an addition of autologous cells.

11. The method according to claim 1, wherein at least a part of the cells in the organoid bodies are living cells during the preparation of the material composition.

12. The method according to claim 1, wherein the cells in the organoid bodies are in a dead state during the preparation of the material composition.

13. The method according to claim 1, wherein the preparation of the material composition comprises a combining of the organoid bodies to form a composite.

14. The method according to claim 13, wherein a plurality of organoid bodies is subjected to at least one of the following steps during combining:
growing together,
mutual adherent adhesion,
compression, and
loading onto or into a carrier device.

15. The method according to claim 13, wherein the composite continues to grow after combining.

16. The method according to claim 15, wherein a form of the composite is adjusted during the preparation by its growth.

17. The method according to claim 1, wherein a form of the composite is adjusted during the preparation by the form of an imprinting device.

18. The method according to claim 17, wherein a cultivating substrate, an imprinting surface or a flexible container is used as the imprinting device.

19. The method according to claim 1, wherein an inner structure of the material composition is adjusted during the preparation.

20. The method according to claim 19, wherein the structure of the material composition is adjusted by an effect of an electrical current.

21. The method according to claim 15, wherein the composite is composed of different differentiated organoid bodies or tissue bodies that grew from different differentiated organoid bodies.

22. The method according to claim 1, wherein an addition of flavoring substances is provided.

23. The method according to claim 1, wherein the stem cells are isolated from glandular tissue of a vertebrate.

24. The method according to claim 23, wherein the stem cells are isolated from glandular tissue of a fish, bird or non-human mammal.

25. A biological material composition produced by a method in accordance with claim 1.

26. A method of feeding an animal, said method comprising feeding to the animal the biological material composition according to claim 25.

27. The method according to claim 26, wherein the biological material composition is a synthetic meat product.

28. The method according to claim 5, wherein the growth of the tissue bodies takes place in a culture medium without an additive that influences a differentiation of cells.

29. The method according to claim 5, wherein the growth to form tissue bodies takes place in a culture medium that contains at least one additive by way of which a plurality of the cells belonging to an organoid body or tissue body is differentiated to a certain cell type.

30. The method according to claim 5, wherein at least a part of the cells in the tissue bodies are living cells during the preparation of the material composition.

31. The method according to claim 5, wherein the cells in the tissue bodies are in a dead state during the preparation of the material composition.

32. The method according to claim 5, wherein the preparation of the material composition comprises a combining of the tissue bodies to form a composite.

33. The method according to claim 32, wherein a plurality of tissue bodies is subjected to at least one of the following steps during combining:
growing together,
mutual adherent adhesion,
compression, and
loading onto or into a carrier device.

34. The method according to claim 32, wherein the composite continues to grow after combining.

35. The method according to claim 32, wherein a form of the composite is adjusted during the preparation by its growth.

36. The biological material composition according to claim 25, further comprising at least one flavoring substance.
